(19) United States
(12) Patent Application Publication
(10) Pub. No.: US 2017/0014174 A1
(43) Pub. Date: Jan. 19, 2017

(54) Cryogenic Cylinder

(71) Applicant: Candela Corporation, Wayland, MA (US)

(72) Inventors: Lewis Levine, Weston, MA (US); Kevin Schomacker, Maynard, MA (US); Walid Kayali, Allston, MA (US); Perry Tomasetti, Pepperell, MA (US)

(21) Appl. No.: 15/194,623

(22) Filed: Jun. 28, 2016

Related U.S. Application Data
(60) Provisional application No. 62/193,610, filed on Jul. 17, 2015.

(51) Int. Cl. A61B 18/02; G05B 15/02

(52) U.S. Cl. CPC A61B 18/02; G05B 15/02; A61B 2018/0262; A61B 2018/00452

(57) ABSTRACT

A cryogenic cylinder including authenticating features and an apparatus recognizing the features and facilitation the cylinder use. The authenticating features prevent use of an unauthorized cryogenic cylinders. The cylinder also includes a number of mechanical features preventing repeat filling of the cylinder by cryogenic fluid or reuse of the cylinder once empty.

The present application claims priority to U.S. provisional application No. 62/193,610 filed Jul. 17, 2015, the contents of which are hereby incorporated by reference into this specification.

### TECHNOLOGY FIELD

The present cryogenic cylinder relates to vessels for storage of liquids and in particular of cryogenic liquids.

### BACKGROUND

Many skin treatment systems include vessels filled with a substance that could be used as a skin cooling material. The skin cooling material can be packed in cartridges, cylinders, canisters and other similar products. Such cylinders or canisters are typically installed by the user and may be removed and replaced by the user, when the skin cooling material in the vessel is depleted.

For example, many of Syneron-Candela skin treatment systems incorporate a Dynamic Cooling Device (DCD) used to provide skin protection and palliative relief during aesthetic skin treatment procedures. The aesthetic system deposits a user selectable amount of cryogen spray prior to, during, or after the aesthetic skin treatment so as to provide these beneficial effects. Cryogen is stored in a vessel under pressure so that it can be delivered to the skin in liquid state.

Typically, each of the materials used for skin treatment is a specially formulated or certified material. For example, the cryogen content of the vessel has to be maintained at a high level of purity. Impurities could result in any or all of the following: patient irritation, allergy, and combustion of impurities during the skin treatment procedure, and restricted flow from impurities clogging the skin cooling material delivery system which may include any or all of the fluid lines, material release valve, and spray jet.

In order to ensure and maintain the purity of the specially formulated or certified cryogenic liquid, the vessel itself needs to not impart contaminants to the liquid. So certifying appropriate vessel materials is needed.

The price of a non-certified cryogenic liquid is usually lower than the price of the specially formulated or certified cryogenic liquid. The users could be tempted to use them at least some of the time, especially when cryogenic fluid vessels are depleted and become available to third parties. Notwithstanding safety issues, this savings in cost is quickly lost when the skin treatment delivery system needs to be replaced because of a clogged skin cooling material delivery system. Potential safety issues from using non-certified cryogenic liquid and/or cryogen cylinder may include: patient irritation, allergy, combustion of impurities during the skin treatment procedure, and ineffective skin cooling during the skin treatment procedure.

### GLOSSARY

As used in the present document the terms vessel, cylinder, canister, have the same meaning, are used interchangeably and designate objects capable of storing a liquid and in particular a cryogenic liquid.

The term skin treatment system as used in the present document includes any skin treatment apparatus including apparatuses for aesthetic skin treatment applying to the skin light energy, radio frequency (RF) energy or ultrasound energy and a combination of the above treatment energies.

As used in the current disclosure the term “authentication” may include any or all of the following: that the vessel and/or cryogen supplied is from a known and authorized source, the filling of the vessel with said cryogen took place under known conditions, and/or the filled vessel was conveyed to the end user by an authorized supplier.

### SUMMARY

An apparatus for aesthetic skin treatment that receives a cryogenic liquid storing cylinder configured to store and dispense a cryogenic liquid. The cryogenic liquid in the container could be under pressure. The cylinder and the apparatus include a number of authenticating features and modules configured to read these features and communicate the information to a processor configured to authenticate one or more features. If the features are authenticated as genuine or certified features, the processor facilitates use of the cylinder and the cryogenic liquid. If at least one feature of the cylinder is not genuine or certified feature, the processor prevents the skin treatment apparatus from dispensing cryogen from the unauthorized cylinder.

### LIST OF FIGURES AND THEIR BRIEF DESCRIPTION

FIG. 1 is a plan view of a cryogenic liquid cylinder in accordance with one example;

FIG. 2 is an example a skin treatment skin receptacle of the cryogenic liquid cylinder of FIG. 1;

FIG. 3 is another example of a cross section of the cryogenic liquid cylinder installation;

FIG. 4 is a schematic block diagram of a skin treatment system using the present cryogenic liquid cylinder in accordance with one example; and

FIG. 5 is an example of cryogenic cylinder authentication process.

### DESCRIPTION

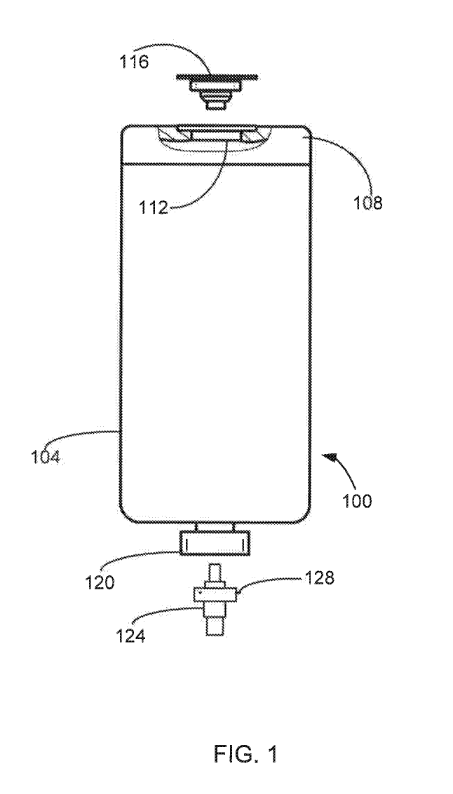

FIG. 1 is a plan view of a cryogenic liquid cylinder or container in accordance with one example. Cryogenic liquid cylinder or container 100 includes a cylindrical body 104 with one or first end of the body terminated by a lid 108 including an opening 112 configured to receive a safety pressure relief valve 116. Although, pressure relief valve 116 is illustrated as a single element, it could be an assembly of a number of parts. Pressure relief valve 116 operates on gaseous materials as liquid states do not respond as readily to pressure changes. Accordingly, the pressure relief valve 116 is located in the part of the cylinder that in working position contains cryogenic gas.

The cryogenic liquid in the container is under pressure. In the present disclosure, the pressure relief valve 116 maintains a safe container or cylinder operating pressure within the container 100.

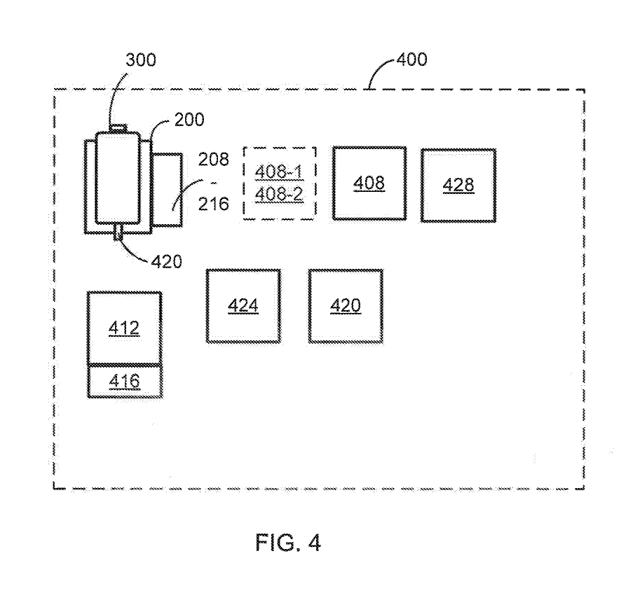

A fitting 120 configured to facilitate connection of cryogenic liquid cylinder 100 to a skin treatment apparatus 400 (FIG. 4) terminates second end of cryogenic liquid cylinder 100. Fitting 120 includes at least a valve 124 that will be termed fitting valve. Fitting valve 124 facilitates inlet of the cryogenic liquid into the liquid containing body or cavity of cryogenic cylinder 100 when the cryogenic cylinder is filled with the cryogenic liquid. The cryogenic liquid in the container is maintained under pressure. Fitting 120 is also configured to facilitate cryogenic liquid from cylinder 100 cavity (not shown) containing the cryogenic liquid discharge.

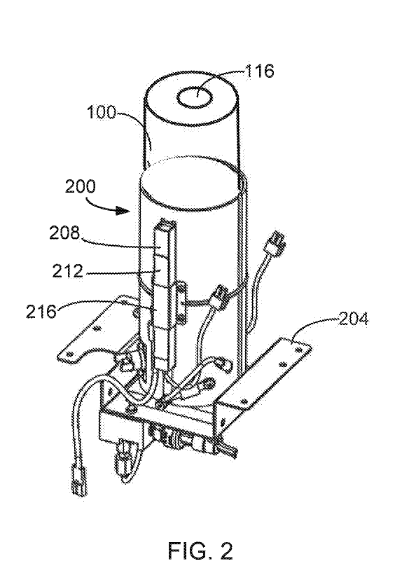

Cylinder 100 and fitting valve 124 could include one or more authentication features. The authenticating features could be mechanical features such as pins 128 (FIG. 1) setting cylinder 100 in a predetermined position. The pins 128 could be of different cross section and located at different height on valve 124. The pins 128 can be appropriately shaped to accommodate connecting cylinder 100 to the cryogen discharged cradle 200 (FIG. 2) as a way to defeat use of unauthorized cylinders. Electronic circuits communicating with the skin treatment apparatus that reads information by a scanner could be another authenticating features. For example, the authenticating features could be such information tags consisting of a UPC code, QR code, color code, encrypted code and a combination of the above. The electronic circuit could be combined with mechanical features such as the authentication pins 128 to further strengthen the authentication process.

In use cylinder 100 is inserted into a cradle 200 (FIG. 2) and with the help of fitting valve 124 connects to cradle 200. Cradle 200 includes a support 204 facilitating connection of the cradle to the skin treatment apparatus (not shown). Cradle 200 also includes a number of cryogenic cylinder or container 100 authentication modules 208-216. The authentication modules 208-216 could be any one of a group of modules consisting of a mechanical authentication module, electronic authentication module, readable information authentication module and a combination of the above. In one example, cylinder 100 could include more than one authenticating feature.

The electronic cylinder authentication module could include RFID tags, RF transponder and/or receiver tags, customized integrated circuits, MEMs configured to perform physical and chemical analyses of the cryogenic liquid that is under pressure, and a mix of the above. The electronic cylinder authentication modules of cradle 200 could communicate with corresponding authentication modules installed in the skin treatment apparatus (not shown). The electronic cylinder authentication modules of cradle 200 could communicate with more than one authenticating feature.

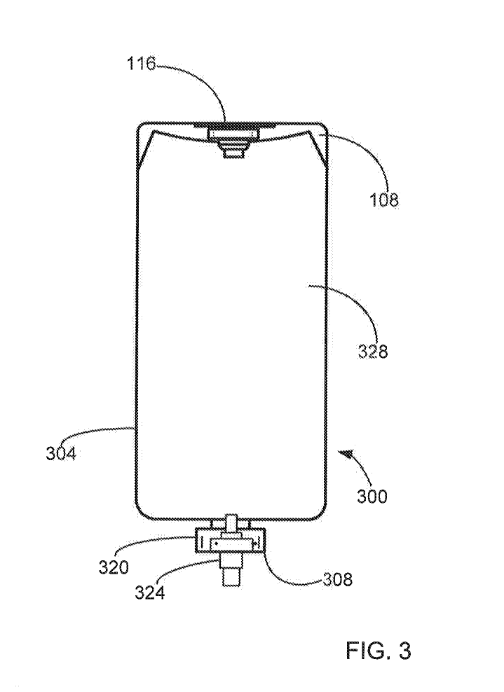

FIG. 3 is of a cross section of an additional example of a cryogenic liquid cylinder. Cryogenic liquid cylinder 300 is similar to cryogenic cylinder 100 and includes a cylindrical body 304 with one or first end of the body terminated by a lid 108 including an opening configured to receive a safety pressure relief valve 116. Although, Pressure Relief Valve 116 is illustrated as a single element, it could be an assembly of a number of parts. Pressure relief valve 116 operates on gaseous materials as liquid states do not respond as readily to pressure changes. Accordingly, the pressure relief valve 116 is located in the part of the cylinder that in working position contains cryogenic gas.

In the present disclosure, the pressure relief valve 116 maintains a safe cryogenic liquid operating pressure within the container 300.

Fitting 320 includes a single use self-locking fitting valve 324 that upon filling of the cylinder cavity 328 by the cryogenic liquid is locked and does not support additional cylinder refill. In case of removal of partially or completely depleted cryogenic cylinder from the skin treatment apparatus, fitting valve 324 is a single use valve and does not support second or additional mounting of the cylinder on the skin treatment apparatus.

In a further example, upon depletion of the cryogenic liquid in cylinder 300 and/or in course of the container from the skin treatment apparatus removal, single use fitting valve 324 is either destroyed or changes its orientation to a position that would prevent cylinder 300 refill by a non-certified cylinder or cryogenic liquid supplier. In one example, fitting valve 324 is pushed into the internal cavity of container 300. As it will be explained below, a certified single use fitting valve 324 could include different authenticating items. The skin treatment apparatus could become not operational when a not certified one-way valve is detected.

Cylinder 300 and single use fitting valve 324 could include one or more authentication features. The authenticating features could be mechanical features such as pins 128 (FIG. 1) setting cylinder 300 in a predetermined position, electronic circuits communicating with the skin treatment apparatus, readable by a scanner information and a combination of the above. For example, the authenticating features could be such information tags consisting of a UPC code, QR code, color code, encrypted code and a combination of the above. More than one authenticating feature could be present and operated simultaneously.

Valve 324 could include one or more authentication features. The authenticating features could be mechanical features, electronic circuits communicating with the skin treatment apparatus, information read by a scanner, and a combination of the above. For example, the authenticating features could be such information tags consisting of a UPC code, QR code, color code, encrypted code and a combination of the above.

Single use fitting valve 324 of cryogenic cylinder or liquid storing container 300 could be configured to facilitate a pass of the cryogenic liquid into the liquid containing cavity 328 of cylinder 300 and delivery of the cryogenic liquid being under pressure from the cylinder. Fitting valve 324 could be also configured to support a least one angular orientation different from a previous angular orientation and a number of different heights with respect to mounting plate 204 or edge 308 of cryogenic cylinder 300.

Cylinder 300 and Valve 324 could include one or more authentication features. The authenticating features could be mechanical features, electronic circuits communicating with the skin treatment apparatus, information read by a scanner, and a combination of the above. For example, the authenticating features could be such information tags consisting of a UPC code, QR code, color code, encrypted code and a combination of the above.

FIG. 4 is a schematic block diagram of a skin treatment apparatus using the present cryogenic liquid cylinder in accordance with one example. Apparatus 400 for aesthetic skin treatment or simply skin treatment includes a cradle 200 configured to receive any one of the liquid storing cylinders 100 or 300 for storing and dispensing the cryogenic liquid contained under pressure in the cylinder. Apparatus 400 includes one or more cylinder or container authentication modules 408 communicating with cryogenic cylinder authenticating modules 208-216, providing information on the authenticity of the cylinder and in some examples of the cryogenic liquid filling the cryogenic cylinder. Authentication module 408 could also be associated with cradle 404 or be a separate unit including a number of cylinder authentication module 408-1, 408-2 etc., for example such as unit 408-1 shown in broken lines. Apparatus 400 further includes a controller 412 configured to communicate with the authentication module 408 of the cryogenic liquid cylinder, valve 324 of the cylinder and in some examples with the module determining content of the cylinder. Controller 412 of the apparatus 400 for aesthetic skin treatment includes a processor circuit 416 configured to receive the information related to the authenticity of the cylinder and in some examples of the cryogenic liquid and compare the received information to an existing database embedded in the apparatus or controller 412 of the apparatus for aesthetic skin treatment. Based on the results of the cylinder authentication, controller 412 or processor circuit 416 could facilitate use of identified/authorized cryogenic liquid cylinder or prevent use of an unauthorized cylinder.

The information provided by the authentication module could be encrypted and the controller 412 could include processes and algorithms supporting decoding of the encrypted information. Processor circuit 416 of skin treatment apparatus 400 decodes the information so that validity of the information conveyed to the skin treatment apparatus could be determined.

Controller 412 can also include algorithms for disabling the authentication modules once the cylinder 300 (100) is depleted of the skin cooling material. Disabling mechanisms can include writing encrypted information onto electronic circuits that notifies controller 412 that the cylinder 300 (100) is no longer authentic or simply erasing the authenticating information such that the cylinder 300 (100) is no longer authenticated. Disabling algorithms can be initiated when the algorithm that determine the remaining volume of skin cooling material falls below a threshold volume, when the pressure in the cylinder 300 (100) falls below a threshold pressure, or when the cylinder 300 (100) is removed from the cradle 200. An example could include release of a detent held in position via the mechanical pins 128 that signals controller 412 to start the disabling algorithm.

Apparatus 400 also includes a communication mechanism 428 between apparatus 400, cryogenic cylinder or container 300 (100) and a remote control computer or control center. If and when insertion of non-authenticated cylinder is detected, communication mechanism 428 could convey this to the remote control computer.

The cryogenic cylinder or container authentication module 208 (212-216) of skin treatment apparatus 400 could be any one of a group of modules consisting of a mechanical authentication module, electronic authentication module, readable information authentication module and a combination of the above. The mechanical cylinder authentication module could include specially keyed mating shapes made in the cradle of the cryogenic cylinder. The shapes could be such as oval shapes, triangular shape, shapes including special cutouts and other shapes.

The electronic cylinder authentication module could include RFID tags, RF transponder and/or receiver tags, customized integrated circuits, MEMs configured to perform physical and chemical analyses of the cryogenic liquid and a mix of the above. Corresponding authentication modules communicating with the authentication modules of the cryogenic cylinder could be installed in apparatus 400.

The cylinder authentication module installed in cradle 200 could include a unit or device configured to read such information tags as UPC code, QR code, color code, encrypted code and a combination of the above.

Apparatus 400 supports on-demand dispensing of the cryogenic liquid contained under pressure in cylinder or container 100 or 300. Generally, each on-demand delivery of the cryogenic liquid could be caused by a push of a button located on the applicator (not shown) or pushing a foot pedal (not shown). The capacity of cryogenic cylinder is known and each on-demand delivery of the cryogenic liquid delivers a known amount of the cryogen liquid. Controller 412 could be also configured to keep record of amount of cryogenic liquid remaining in the cylinder. Controller 412 could also be configured to keep record of the amount of cryogenic liquid dispensed from the cylinder 100 (300).

Cradle 200 of apparatus 400 (FIG. 4) in addition to receiving and holding the cryogenic liquid cylinder 300 (100) includes a mechanism 420 configured to destroy by locking, or pushing inside the cylinder single use fitting valve 324 supporting filling of the cylinder by the cryogenic liquid and discharge of the cryogenic liquid being maintained under pressure, from the cylinder or container 300. For example, the fitting valve 324 (128) can be rotated by an angle such as 90 degrees when the empty cylinder 300 (100) is removed from apparatus 400 thereby locking the fitting valve 324 (128) in place such that the fitting valve 324 (128) cannot open. As a result cylinder 300 (100) cannot be refilled. In this example, a partially empty cylinder 300 is also disabled when removed from cradle 200.

In another example, the single use fitting valve 324 (128) can be rotated by an angle such as 90 degrees when the empty cylinder 300 (100) is removed from apparatus 400. In this position, the single use fitting valve 324 (128) is held in place only by the pressure within cylinder 300 (100). Thereby once cylinder 300 (100) is empty, there is insufficient pressure in cylinder 300 (100) to hold fitting valve 324 (128) in place causing it to fall into cylinder 300 (100). As a result fitting valve 324 (128) is no longer effective and canister 300 (100) cannot be refilled. In this example, a partially empty cylinder 300 provided there is sufficient pressure is still enabled when removed from cradle 200.

In another example, the single use fitting valve 324 (128) can be rotated by an angle such as 90 degrees when the empty cylinder 300 (100) is removed from apparatus 400. In this position, the single use fitting valve 324 (128) is held in place only by the pressure within cylinder 300 (100). Thereby once cylinder 300 (100) is empty, there is insufficient pressure in cylinder 300 (100) to hold fitting valve 324 (128) in place when connecting to the cryogen fill station filling hose causing it to fall into cylinder 300 (100). As a result fitting valve 324 (128) is no longer effective and canister 300 (100) cannot be refilled.

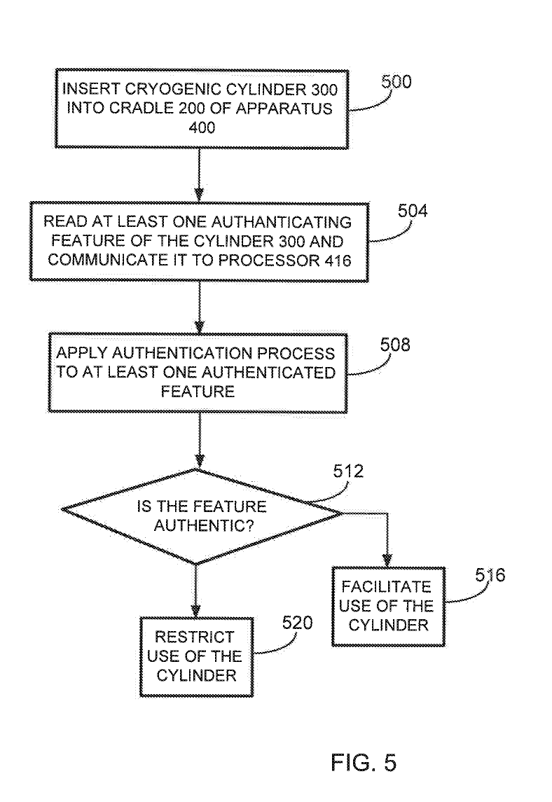

FIG. 5 is an example of cryogenic cylinder or canister authentication process. Following the insertion of the cryogenic cylinder 100 or 300 into cradle 200 of a skin treatment apparatus 400 (Block 500), a reading device reads at least one authenticating feature of the cryogenic cylinder 100 or 200 and communicates the read information to processor circuit 416 (Block 504). Processor circuit 416 applies authentication process or algorithm to at least one authenticated feature (Block 508) received by the processor circuit 416 and then determines authenticity of at least one authenticated feature (Block 512). If all required cryogenic cylinder features are determined as authentic features, processor circuit 416 facilitates use of the inserted cryogenic cylinder (Block 516) by apparatus 400. If at least one feature of the cryogenic cylinder could not be identified as authentic feature, processor circuit 416 could alert the caregiver through audio means or an informational prompt on the system to the presence of unauthorized vessels or content. Processor circuit 416 could also disable or restrict use of the cryogenic cylinder (Block 520) by the skin treatment apparatus 400.

Processor circuit 416 could also be configured to record in a non-volatile memory circuit 424 of apparatus for aesthetic skin treatment 400 (FIG. 4) that a non-authorized or non-authenticated cryogenic liquid cylinder has been installed and used by apparatus 400. Apparatus 400 could also record the amount of non-authenticated cryogenic liquid that apparatus 400 has consumed. The record could be stored locally in the apparatus or communicated to a remote control computer. Processor 400 could be further configured to restrict the use of non-authenticated cylinder 100 (300), or disable the use of non-authenticated cylinder 100 (300), or permanently disable use of cryogen cradle 200 until the system is inspected by a qualified service engineer.

The operations and algorithms described herein can be implemented as executable code within the micro-controller or controller 412 having processor circuit 416 as described, or stored on a standalone computer or machine readable non-transitory tangible storage medium that are completed based on execution of the code by a processor circuit implemented using one or more integrated circuits. Example implementations of the disclosed circuits include hardware logic that is implemented in a logic array such as a programmable logic array (PLA), a field programmable gate array (FPGA), or by mask programming of integrated circuits such as an application-specific integrated circuit (ASIC). Any of these circuits also can be implemented using a software-based executable resource that is executed by a corresponding internal processor circuit such as a micro-processor circuit (not shown) and implemented using one or more integrated circuits, where execution of executable code stored in an internal memory circuit causes the integrated circuit(s) implementing the processor circuit to store application state variables in processor memory, creating an executable application resource (e.g., an application instance) that performs the operations of the circuit as described herein. Hence, use of the term “circuit” in this specification refers to both a hardware-based circuit implemented using one or more integrated circuits and that includes logic for performing the described operations, or a software-based circuit that includes a processor circuit (implemented using one or more integrated circuits), the processor circuit including a reserved portion of processor memory for storage of application state data and application variables that are modified by execution of the executable code by a processor circuit. The memory circuit 424 can be implemented, for example, using a non-volatile memory such as a programmable read only memory (PROM) or an EPROM, and/or a volatile memory such as a DRAM, etc.

Use of specially formulated or certified for skin treatment materials and in particular cryogenic liquids could avoid any or all of the following: patient irritation, allergy, combustion of impurities during the skin treatment procedure, and ineffective skin cooling due to restricted skin cooling material flow from impurities clogging the skin cooling material delivery system which may include any or all of the fluid lines, material release valve, and spray jet.

What is claimed is:

1. A cryogenic liquid storing cylinder comprising:
a liquid containing body configured to contain a liquid under pressure;
a fitting configured to include a single use fitting valve facilitating inlet of the liquid into the liquid containing body of the cylinder;
wherein the single use fitting valve is also configured to facilitate dispensing the liquid from the liquid containing body of a liquid storing cylinder; and
wherein the single use fitting valve is configured to be destroyed upon depletion of the liquid in the cylinder.

2. The liquid storing cylinder according to claim 1 wherein the single use fitting valve falls inside the container upon depletion of the liquid in the container.

3. The liquid storing cylinder according to claim 1 wherein the cylinder further includes a pressure relief valve; and wherein the pressure relief valve operates on a gaseous aggregate state of the liquid.

4. The liquid storing cylinder according to claim 1 wherein the cylinder includes at least one cylinder authenticating feature associated with the cylinder.

5. The liquid storing cylinder according to claim 1 further comprising an electronic module located at inlet of the liquid into the container.

6. The liquid storing cylinder according to claim 1 further comprising an electronic module located in the fitting valve facilitating inlet of the liquid into the liquid containing body of the cylinder.

7. The liquid storing cylinder according to claim 1 wherein the fitting valve includes at least one of a group of authentication features consisting of a mechanical feature, electronic circuit, readable information and a combination of the above.

8. An apparatus for aesthetic skin treatment configured to receive at least one cryogenic liquid storing cylinder for storing and dispensing a cryogenic liquid including:
a cylinder authentication module providing information on at least one of the authenticity of the cryogenic liquid cylinder and cryogenic liquid filling the cylinder;
at least one controller configured to communicate with at least one identification feature of the cryogenic liquid cylinder and content of the cylinder; and
wherein at least one control mechanism is further configured to keep record of at least one of amount of cryogenic liquid remaining in the cylinder and amount of cryogenic liquid dispensed from the cylinder.

9. The apparatus according to claim 8 wherein the cryogenic cylinder authentication module is at least one of a group of modules consisting of a mechanical module, electronic module, readable information module and a combination of the above.

10. The apparatus according to claim 8 wherein a mechanical cylinder authentication module includes specially keyed mating pins made in the cryogenic cylinder and in apparatus receiving the cylinder.

11. The apparatus according to claim 8 wherein electronic cylinder authentication module is at least one of a group of modules consisting of RFID tags, customized integrated circuits, physical and chemical analyses of the cryogenic liquid and a combination of the above.

12. The apparatus according to claim 8 wherein readable information cylinder authentication module is at least one of a group of modules configured to read at least one of a group of information tags consisting of a UPC code, QR code, color code, encrypted code and a combination of the above.

13. The apparatus according to claim 8 wherein the controller configured to communicate with the cryogenic liquid cylinder and content of the cylinder is configured to communicate with at least one of authentication modules and facilitate cylinder use.

14. The apparatus according to claim 8 wherein the controller is also configured to provide information indicating at least one of authenticity and suitability of the cylinder and cryogenic liquid filling the cylinder.

15. The apparatus according to claim 8 wherein the controller is also configured to provide information indicating amount of at least one of authenticity and suitability of the cylinder and cryogenic liquid filling the cylinder.

16. The apparatus according to claim 8 further comprising a cryogenic liquid cylinder cradle configured to receive and hold the cylinder in course of apparatus operation and further configured to destroy a fitting valve supporting filling of the cylinder by the cryogenic liquid.

17. The apparatus according to claim 8 wherein the information module conveys the information to a skin treatment apparatus in encrypted form and wherein the skin treatment apparatus (processor) decodes the information so that validity of the information conveyed to the skin treatment apparatus could be determined.

18. The apparatus according to claim 8 wherein the information module disables at least one of the cryogenic cylinder authentication modules when cryogenic cylinder is depleted.

19. The apparatus according to claim 8 wherein the apparatus further includes a communication mechanism between apparatus and a remote control computer or control center.

20. The apparatus according to claim 19 wherein the communication mechanism communicates at least with remote control computer to convey a message that non-authenticated cylinder inserted in the apparatus has been detected.

21. An authentication method for authenticating a cryogenic cylinder inserted in a skin treatment device, comprising:
inserting a cryogenic cylinder filled with a cryogenic liquid into a cradle of a skin treatment apparatus;
reading at least one authenticating feature of the cryogenic cylinder and communicating it to a processor;
applying an authentication process to at least one authenticated feature received by the processor and determining authenticity of at least one authenticated feature; and
disabling or aborting use of the cryogenic cylinder by the skin treatment apparatus if the cryogenic cylinder is not authentic.
